(19) United States
(12) Patent Application Publication
(10) Pub. No.: US 2010/0210023 A1
(43) Pub. Date: Aug. 19, 2010

(54) Salivary metabolic biomarkers for human oral cancer detection

(75) Inventors: David T.W. Wong, Beverly Hills, CA (US); Masaru Tomita, Tsuruoka (JP); Masahiro Sugimoto, Tsuruoka (JP); Akiyoshi Hirayama, Tsuruoka (JP); Tomoyoshi Soga, Tsuruoka (JP)

Correspondence Address: OLIFF & BERRIDGE, PLC, P.O. BOX 320850, ALEXANDRIA, VA, 22320-4850, US

(73) Assignees: THE REGENTS OF THE UNIVERSITY OF CALIFORNIA, OAKLAND, CA (US); KEIO UNIVERSITY, TOKYO (JP)

(21) Appl. No.: 12/585,598

(22) Filed: Sep. 18, 2009

Related U.S. Application Data
(60) Provisional application No. 61/099,110, filed on Sep. 22, 2008.

(51) Int. Cl. G01N 33/68; G01N 33/00; G01N 27/26; B01D 57/02

(52) U.S. Cl. 436/90; 436/106; 436/127; 436/111; 436/96; 205/792; 204/451

(57) ABSTRACT

The present invention provides a novel oral cancer and periodontal disease salivary metabolome for use in the diagnosis or for providing a prognosis for oral cancer and periodontal disease in an individual. The present invention also provides novel methods of diagnosing or providing a prognosis for oral cancer or periodontal disease by detecting metabolites found in the saliva of an individual. Finally, the present invention provides kits for the detection of salivary metabolites useful in the diagnosis or prognosis of oral cancer and periodontal disease in an individual.

### CROSS-REFERENCES TO RELATED APPLICATIONS

This application claims the benefit of priority to U.S. provisional application U.S. 61/099,110, filed on Sep. 22, 2008, which is incorporated herein by reference.

### STATEMENT AS TO RIGHTS TO INVENTIONS MADE UNDER FEDERALLY SPONSORED RESEARCH AND DEVELOPMENT

This invention was made with Government support under NIH Grant No. RO1 DE 015970. The Government has certain rights in this invention.

### REFERENCE TO A “SEQUENCE LISTING,” A TABLE, OR A COMPUTER PROGRAM LISTING APPENDIX SUBMITTED ON A COMPACT DISK

Not Applicable

### BACKGROUND OF THE INVENTION

Oral cancer, predominantly oral squamous cell carcinoma (OSCC), is a high impact disease in the oral cavity, affecting more than 34,000 people in the United States each year (American Cancer Society, 2007) and more than 400,000 people annually worldwide (The Oral Cancer Foundation www.oralcancerfoundation.org). Despite treatment advances, the disease's overall 5-years survival rate is only about 50% and has not improved in the past 30 years, remaining among the worst of all cancers (Epstein, J. B. et al., J Can Dent Assoc, 68(10):617-21 (2002); Mao, L. et al., Cancer Cell, 5(4):311-16 (2004)). The death rate associated with this cancer is particularly high not because it is hard to discover or diagnose, but due to the cancer being routinely discovered late, after metastasis has already spread to the lymph nodes or the neck (The Oral Cancer Foundation www.oralcancerfoundation.org). Thus, a novel, non-obtrusive diagnostic tool that is inexpensive, highly sensitive, and accurate, is necessary to detect oral cancer early as possible in order to reduce the high mortality rate (Mignogna, M. D. et al., Eur J Cancer Prev, 13(2):139-42 (2004)).

Oral cancer tumors arise through a series of molecular mutations that lead to uncontrolled cellular growth from hyperplasia to dysplasia to carcinoma in situ followed by invasive carcinoma. Major risk factors include tobacco and alcohol consumption along with environmental and genetics factors (Brinkman and Wong, 2006; Figuerido et al., 2004; Hu et al., 2007; Turhani et al., 2006). These cancers are usually detected at late stages when the disease has advanced and therefore results in poor prognosis and survival. Presently, surgery and radiotherapy are the primary treatments, but due to the location in the head and neck; this usually results in postoperative defects and functional impairments in patients (Thomson and Wylie, 2002). Therefore, early disease detection is imperative because it can result in a more effective treatment with superior results.

Saliva, blood, and urine are systematically affected by various pathways and therefore these biofluids are well suited for use in monitoring systemic diseases and conditions. A number of clinical tools have been developed to aid in cancer diagnosis and prognosis through the detection of differential gene expression in serum and urine (Drake, R. R. et al., Expert Rev Mol Diagn, 5(1):93-100 (2005); Hu, S. et al., Expert Rev Proteomics, 4(4):531-38 (2007)). Although saliva-based detection of early stage oral cancer has been hampered in the past due to the low density of saliva, low variety of associated genetic mutations, and various effects caused by cancer progress, a number of studies have explored the molecular level of markers that discriminate between patients with oral cancer and healthy individuals. For example, concentrations of Cyfra 21-1, tissue polypeptide antigen, CA125, CA19-9, SCC, and carcinoembryonic, tumor markers of oral squamous cell carcinoma (OSCC) in serum, have also been found to be elevated in the saliva of OSCC patients (Nagler, R. et al., Clin Cancer Res, 12(13):3979-84 (2006)).

Saliva has gained notable attention as a diagnostic fluid because of its simple collection and processing, minimal invasiveness and low costs. Many researchers have studied salivary proteins as potential diagnostic markers for various diseases such as breast cancer, ovarian cancer, Sjögrens syndrome, hepatocellular carcinoma, leukoplakia and oral cancer (Ryu et al., 2006; Streckfus et al., 2000; Rhodus et al., 2005; Brailo et al., 2006, Yio et al., 1992; Gorelik et al., 2005; Hu et al., 2007). The human salivary proteome was recently completed and it was found that salivary ductal fluids from the parotid, submandibular, and sublingual glands contain roughly 1,166 proteins (Denny et al., J. Proteome Res., 7(5):1994-2006 (2008)). Use of these proteins as potential salivary disease markers may lead to the development of simple clinical tools for early detection of numerous diseases as well as for monitoring disease progression before, after, and during treatment (Kingsmore, 2006).

Other examples of studies identifying salivary markers include Xie et al., who identified over one thousand proteins in the whole saliva of oral cancer patients. Their analysis revealed that whole saliva contains exfoliated cells, the majority of which are epithelial, as well as over 30 different bacterial species, some of which putatively contribute to cancer development (Xie, H. et al., Mol Cell Proteomics, 7(3):486-98 (2008)). Shpitzer et al. analyzed inorganic compounds and proteins such as albumin, lactate dehydrogenase, amylase, total immunoglobulin, etc. in the saliva of oral squamous cell carcinoma (OSCC) patients. They found that potassium and amylase levels were significantly reduced while the level of other markers were increased in the saliva of oral cancer patients as compared to control samples (Shpitzer, T., et al., J Cancer Res Clin Oncol, 133(9):613-17 (2007)). Chen et al. found that levels of both alpha-amylase and albumin were significantly higher in the saliva of OSCC patients than in normal controls, as measured by matrix-assisted laser desorption/ionization mass spectrometry (MALDI-MS) (Chen, Y. C. et al., Rapid Commun Mass Spectrom, 16(5):364-9 (2002)). Li et al. developed a transcriptome diagnostic approach for oral cancer detection based on mRNA profile pattern analysis. Classification and regression trees (CART) model containing quantitative levels of RNA, OAZ, SAT, IL8, IL1b resulted in a diagnostic method with greater than 90% sensitivity and specificity for distinguishing patients with OSCC from the control individuals (Li, Y. et al., Clin Cancer Res, 10(24):8442-50 (2004)). Finally, Hu et al. used MALDI-MS in a shotgun proteomics approach to discover a panel of five highly discriminatory salivary proteins that are found at significantly different levels in the saliva specimen from OSCC patients as compared to controls (Hu, S. et al., Cancer Genomics Proteomics, 4(2):55-64 (2007)) (Hu et al., 2008).

Metabolics hold the potential for bridging the gap between genotype and phenotype as well as for promoting comprehensive and holistic understanding of a cell. Metabolic methodologies enable simultaneous monitoring of many hundreds of metabolites in both qualitative and quantitative fashion and allow for the elucidation of cellular pathway behaviors that result in response to specific environmental variances (Fernie, A. R. et al., Nat Rev Mol Cell Biol, 5(9):763-9 (2004); Fraenkel, D. G., Annu Rev Genet, 26:159-77 (1992); Ideker, T. et al., Science, 292(5518):929-34 (2001); Raamsdonk, L. M. et al., Nat Biotechnol, 19(1):45-50 (2001); Spinnler, H. E. et al., Proc Natl Acad Sci USA, 93(8):3373-6 (1996)). Large scale metabolite analysis have been performed using gas chromatography coupled with mass spectrometry (Fiehn, O. et al., Nat Biotechnol, 18(11):1157-61 (2000)), liquid chromatography coupled with mass spectrometry (LC-MS) (Plumb, R. et al., Analyst, 128(7):819-23 (2003)), NMR (Reo, N. V., Drug Chem Toxicol, 25(4):375-82 (2002)), Fourier transform ion cyclotron resonance mass spectrometry (Aharoni, A. et al., Omics, 6(3):217-34 (2002)), and capillary electrophoresis coupled with mass spectrometry (CE-MS) (Soga, T. et al., J Proteome Res, 2(5):488-94 (2003)). In two studies, metabolite profiles have been elucidated from the urine of renal cell carcinoma patients (Kind, T. et al., Anal Biochem, 363(2):185-95 (2007); Perroud, B. et al., Mol Cancer, 5:64 (2006)). Metabolome analysis by NMR has also been implemented as a diagnostic tool for the prognosis of lymph node metastasis and for monitoring patient response to chemotherapy for breast cancer (reviewed, Claudino, W. M. et al., J Clin Oncol, 25(19):2840-6 (2007)).

The present invention fulfills a need in the art for salivary metabolic biomarkers useful for diagnosing and providing a prognosis for oral cancers and periodontal disease. The present invention also provides non-invasive methods for the diagnosis of oral cancers and periodontal disease.

### BRIEF SUMMARY OF THE INVENTION

The present invention provides for the first time salivary metabolite biomarkers, identified from global metabolome profiling analysis, for use in oral disease diagnosis and prognosis. Due to the identification of this oral disease metabolome and the advancements in high-throughput capillary electrophoresis-mass spectrometry (CE-MS) techniques, the methods of the present invention are well suited for use in the early detection of oral diseases, including oral cancers and periodontal disease. Furthermore, the methods of the present invention can be used as a discovery platform for the identification of other salivary metabolic biomarkers.

In one embodiment, the present invention provides salivary metabolite biomarkers useful in the diagnosis and prognosis of oral disease in a subject. The small molecule biomarkers found in Table 2 allow for the differentiation of salivary samples from individuals with oral disease, and healthy individuals. In one embodiment, the present invention provides salivary oral disease metabolomes for use in the diagnosis and prognosis of oral disease in a subject. In a specific embodiment, these metabolomes comprise subsets of metabolites found in Table 2. In another embodiment, the metabolomes of the present invention comprise all of the metabolites found in Table 2.

In a second embodiment, the present invention provides methods of diagnosing or providing a prognosis for oral disease in a subject. In certain embodiments, these methods comprise the steps of first determining the level of at least one salivary oral disease metabolite biomarker in a sample of saliva from the subject, then comparing said level to a salivary oral disease metabolome, or both, and finally determining if the at least one salivary oral disease metabolite is present at a differential level in the salivary sample as compared to a level found in a sample from an individual not suffering from oral disease, thereby diagnosing or providing a prognosis for oral disease in the subject. In other embodiments, the level of the at least one salivary oral disease metabolite is compared to a control salivary metabolome. In yet other embodiments, the at least one salivary oral disease metabolites comprise metabolites selected from those found in Table 2.

In a third embodiment, the present invention provides methods of identifying novel salivary oral disease small molecule biomarkers or oral disease metabolite biomarkers. In one embodiment, the methods comprise the steps of first separating metabolites from a salivary sample from one or more individuals suffering from oral disease, then determining the level of said metabolites, and finally determining if any of the metabolites are present at a differential level in the saliva of at least one individual suffering from oral disease as compared to the level of said metabolite in a salivary sample from an individual not suffering from oral disease, thereby identifying a novel salivary oral disease small molecule biomarker.

In a fourth embodiment, the present invention provides methods of developing a model for the classification of disease states in an individual. In one embodiment, the method comprises the steps of first determining the level of at least one metabolite in a first sample of saliva from an individual or group of individuals suffering from a first disease state, next determining the level of said at least one metabolite in a second sample of saliva from an individual or group of individual suffering from a second disease state, and finally comparing the levels of said at least one metabolite from said first and said second sample, thereby developing a model for the classification of said first and said second disease states.

In a fifth embodiment, the present invention provides a method of classifying of differentiating a disease state in an individual suspected of having one of two or more disease states. In some embodiments, the method comprises the steps of determining a salivary metabolic profile from an individual, comparing said metabolic profile to a classification model of said two or more disease states, and determining which disease state most highly correlates with said metabolic profile, thereby classifying the disease state of an individual.

In a sixth embodiment, the present invention provides kits useful in the diagnosis and prognosis of oral disease in an individual. In some embodiments, the kits of the present invention comprise reagents that bind to at least one salivary oral disease metabolite biomarker. In certain embodiments the salivary oral disease biomarkers are those found in Table 2. In other embodiments, the kits comprise a plurality of reagents that bind to a subset of or all of the metabolites found in Table 2.

### BRIEF DESCRIPTION OF THE DRAWINGS

FIG. 1. show a heatmap of 45 peaks showing differential levels (p<0.05) in individuals with oral cancer, healthy control individuals, and individuals with periodontal disease, generated with the TM4 software package.

FIG. 2. shows a result of ROC curve analysis for the discriminating ability of multiple salivary metabolites for A) oral cancer (n=69) or B) periodontal diseases (n=11) between healthy controls (n=87). Solid lines and dotted lines are ROC curves obtained using whole data as training set and 10-fold cross validation, respectively. Using a cutoff probability of 50%, the calculated area under the ROC curves were 0.865 (0.810) for oral cancer and 0.969 (0.954) for periodontal diseases, respectively. NOTE: Non-parenthetic values are obtained by full-training data and parenthetic values are obtained in tenfold cross-validation.

FIG. 3 shows score plots of principal components (PC) analyses. The subjects in all groups are shown in 3-dimensional (a) without three outliers in oral cancer, and its enlarged view (b). The dots denote healthy controls (open circle), oral (open rectangle), breast (christcross), pancreatic cancer (filled circle), and periodontal disease (open triangle). The cumulative proportions of the first, second and third PCs (PC1, PC2, and PC3) were 44.8, 57.6 and 67.0%.

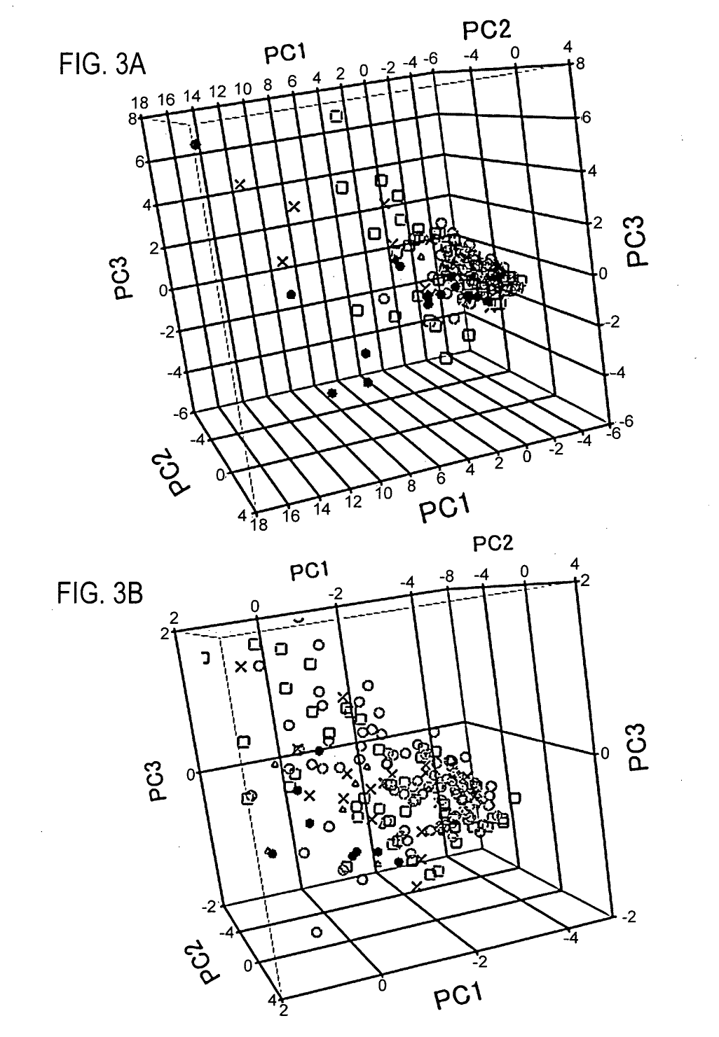

FIG. 4 shows score plots of principal components (PC) analysis according to sex. The values for males and females are visualized in open circle and filled rectangle colored, respectively. Three-dimensional PC plots for healthy controls (a), and for subjects with oral cancer (b) are shown. A total of 42 male (blue) and 27 female (red) control subjects and 41 male and 23 female patients with oral cancer were included in this analysis. Eighteen control subjects, 5 patients with oral cancer and all of the patients with other diseases were excluded because of unavailable sex information. The cumulative proportions of PC1, PC2, and PC3 for the control subjects were 43.0%, 52.9% and 60.6%, respectively, and those for patients with oral cancer were 50.2%, 65.7% and 77.5%, respectively.

FIG. 5. shows principal components (PC) analysis score plots for race and ethnic groups. The data for African-American, Asian, Caucasian and Hispanic subjects are visualized in open circle, filled rectangle, cross joint, and filled circle, respectively. Three-dimensional PC plots for healthy controls (a) and for subjects with oral cancer (b) are shown. The race or ethnic groups included 12 and 4 African-Americans, 15 and 5 Asians, 37 and 41 Caucasians, and 5 and 5 Hispanics as healthy controls and subjects with oral cancer, respectively. Eighteen control subjects, 5 patients with oral cancer, and all of the patients with other diseases were excluded because of unavailable race or ethnic information. The cumulative proportions of PC1, PC2 and PC3 for the control subjects were 43.0%, 52.9% and 60.9%, respectively, and those for subjects with oral cancer were 42.0%, 63.2% and 70.2%, respectively.

### DETAILED DESCRIPTION OF THE INVENTION

Although transcriptomic (Zimmermann, B. G. et al., Ann N Y Acad Sci, 1098:184-91 (2007); Zimmermann, B. G. and Wong, D. T., Oral Oncol, p. doi:10.1016/j.oraloncology.2007.09.009 (2007)) and proteomic studies (Hu, S. et al., Proteomics, 5(6):1714-28 (2005)) of oral cancer have identified potential nucleic acid and protein biomarkers for clinical application, global changes in the levels of salivary metabolites from patients suffering from oral disease have yet to be investigated via metabolic approaches. The present invention provides novel CE-MS methods well-suited for metabolic studies, particularly when high resolution compound separation and high detection sensitivity are required (Soga, T. et al., J Proteome Res, 2(5):488-94 (2003); Soga, T. et al., J Biol Chem, 281(24):16768-76 (2006)). Capillary electrophoresis (CE) enables temporal separation of components based on charge and shape, while mass spectroscopy (MS) provides additional secondary separation for compounds that co-migrate in CE. Thus, CE-MS is particularly suited for salivary metabolome analysis since saliva is known to contain various electrolytes. In one embodiment, the present invention provides small molecule biomarkers useful for the diagnosis and prognosis of oral cancer and periodontal disease, that have been identified by global metabolite profile analysis using CE-MS on salivary samples from patients suffering from oral cancer or periodontal disease. These profiles were compared to profiles generated from salivary samples obtained from healthy control individuals. In a second embodiment, the present invention also provides methods that are complementary approaches for early detection of oral cancers that may be used in conjunction with proteome and transcriptome-based diagnostic tests.

A large-scale metabolic analysis was conducted to explore the differences in the salivary metabolite profiles between healthy individuals and those with oral cancer and periodontal disease. CE-TOF-MS-based analysis identified 28 metabolites that were present in the saliva of patients with oral disease at statistically significant differential levels as compared to those in healthy control individuals (Table 2).

In one embodiment, the present invention provides salivary oral cancer metabolite biomarkers useful for diagnosing and providing a prognosis of oral cancer in a subject. Useful biomarkers include those that are present at a differential level or concentration in the saliva of an individual or group of individuals suffering from oral cancer as compared to the level or concentration in the saliva of an individual or group of individuals not suffering from oral cancer. In one embodiment, the biomarkers comprise the metabolites found in Table 2. In a specific embodiment, the biomarkers are selected from the group consisting of C₈H₉N (120.0801 m/z), Threonine, Leucine, Isoleucine, Cadaverine, Glutamic acid, Tyrosine, Piperideine, alpha-Aminobutyric acid, Serine, Alanine, Valine, Phenylalanine, Pipecolic acid, Choline, C₄H₉N (72.0813 m/z), Tryptophan, C₆H₆N₂O₂ (139.05 m/z), Glutamine, C₅H₁₄N₅ (145.1331 m/z), beta-Alanine, Carnitine, C₆H₈OS₂ or C₄H₅N₂O₁₁P (288.9691 m/z), Piperidine, Pyrroline hydroxycarboxylic acid, Taurine, and Betaine.

In a second embodiment, the present invention provides salivary periodontal metabolite biomarkers useful for diagnosing or providing a prognosis of periodontal disease in a patient. In one embodiment, salivary periodontal disease biomarkers comprise metabolites that are present at a different level or concentration in the saliva of a patient or group of patients suffering from periodontal disease as compared to the level in an individual or group of individuals not suffering from periodontal disease. In another embodiment, the biomarkers comprise those found in Table 2. In a specific embodiment, the periodontal biomarkers of the present invention are selected from the group consisting of C₂H₆N₂ (59.0616 m/z), C₇H₈O₃S (173.0285 m/z), C₃₀H₆₂N₁₉O₂S₃ (409.2312 m/z), C₈H₉N (120.0801 m/z), Threonine, Leucine, Isoleucine, Cadaverine, Putrescine, Tyrosine, Ethanolamine, Piperideine, alpha-aminobutyric acid, Serine, Alanine, Valine, Phenylalanine, Pipecolic acid, Taurine, Tryptophan, Glycerophosphocholine, and gamma-Aminobutyric acid.

In a third embodiment, the present invention provides metabolite biomarkers that are useful for distinguishing between oral cancer and periodontal disease. In one embodiment, the biomarkers comprise small molecules that are present at a different level or concentration in a patient or group of patients suffering from oral cancer as compared to the level or concentration of the metabolite in a patient or group of patients suffering from periodontal disease. In a certain embodiment, the biomarkers are those found in Table 2. In a specific embodiment the biomarkers are selected from the group consisting of C₂H₆N₂ (59.0616 m/z), C₇H₈O₃S (173.0285 m/z), C₈H₉N (120.0801 m/z), Piperideine, Pipecolic acid, C₄H₉N (72.0813 m/z), Taurine, Glycerophosphocholine, and Pyrroline hydroxycarboxylic acid.

In another embodiment, the present invention provides salivary oral cancer metabolomes useful for diagnosing or providing a prognosis for oral cancer in a subject. In one embodiment, the oral cancer metabolomes of the present invention comprise subsets of the metabolites found in Table 2. In other embodiments, the metabolomes comprise at least about 1, 2, 3, 4, 5, 6, 7, 8, 9, 10, 15, 20, 25, 30, 35, 40 or all of the metabolites found in Table 2. In yet other embodiments, the metabolomes comprise a group of metabolites that are present at a differential level or concentration in the saliva of an individual or group of individuals suffering from oral cancer as compared to the level or concentration in the saliva of an individual or group of individuals not suffering from oral cancer. In certain embodiments, the metabolomes of the present invention are specific for a type of oral cancer, such as oral squamous cell carcinoma, lip cancer, tongue cancer, gingival carcinoma, buccal mucosal carcinoma, a head and neck squamous cell carcinoma, and the like. In yet other embodiments, the invention provides salivary metabolomes that correspond to a particular stage or classification of oral cancer, a particular prognosis for oral cancer, a particular prognosis for the course of a treatment for oral cancer, or for a particular prognosis for the efficacy or response of a particular chemotherapeutic drug.

In a related embodiment, the present invention provides salivary periodontal disease metabolomes useful for diagnosing or providing a prognosis for periodontal disease in an individual. In one embodiment, the oral cancer metabolomes of the present invention comprise subsets of the metabolites found in Table 2. In other embodiments, the metabolomes comprise at least about 1, 2, 3, 4, 5, 6, 7, 8, 9, 10, 15, 20, 25, 30, 35, 40 or all of the metabolites found in Table 2. In yet other embodiments, the metabolomes comprise a group of metabolites that are present at a differential level or concentration in the saliva of an individual or group of individuals suffering from periodontal disease as compared to the level or concentration in the saliva of an individual or group of individuals not suffering from periodontal disease. In certain embodiments, the metabolomes of the present invention are specific for a type of periodontal disease, such as gingivitis, gingival periodontitis, cementum periodontitis, alveolar periodontitis, connective tissue periodontitis, and the like. In yet other embodiments, the invention provides salivary metabolomes that correspond to a particular stage or classification of periodontal disease, a particular prognosis for periodontal disease, a particular prognosis for the course of a treatment for periodontal disease, or for a particular prognosis for the efficacy or response of a particular drug used to treat periodontal disease.

In yet another embodiment, the metabolomes provided by the invention are useful for classifying, distinguishing, or differentiating between an oral cancer and a non-oral cancer, an oral cancer and periodontal disease, or periodontal disease and a non-oral cancer. In specific embodiments, the non-oral cancer may be breast cancer or pancreatic cancer. In further embodiments, the metabolomes of the present invention are useful in the development of classification or differentiation models.

In one embodiment of the present invention, methods of identifying novel salivary oral cancer metabolite biomarkers are provided. In a specific embodiment, the methods comprise the steps of: (a) determining the level of a metabolite in a first saliva sample from an individual or group of individuals with oral cancer; (b) comparing the level of said biomarker to the level of the same biomarker in a second saliva sample from an individual or group of individuals without oral cancer; and (c) determining if the level of the metabolite in said first sample is different from the level of the metabolite in said second sample, thereby identifying a salivary oral cancer metabolite biomarker. In another embodiment, the present invention provides methods of identifying novel salivary periodontal disease metabolite biomarkers.

In another embodiment, the present invention provides methods of diagnosing or providing a prognosis for oral cancer in an individual. In a specific embodiment, the methods comprise the steps of: (a) determining the level in a salivary sample from the individual of at least one salivary oral cancer metabolite biomarker selected from those found in Table 2; and (b) determining if the level of said at least one metabolite corresponds to a level found in an oral cancer metabolome profile, thereby diagnosing or providing a prognosis for oral cancer. In particular embodiments, methods of diagnosing or providing a prognosis for oral cancer comprise the steps of (i) comparing the level of said at least one metabolite to a first profile comprising an oral cancer metabolic profile; (ii) comparing the level of said at least one metabolite to a second profile comprising a control metabolic profile; and (iii) determining which metabolic profile is most similar to the level of said at least one metabolite from said individual, thereby diagnosing said individual as either having or not having oral cancer. In yet other embodiments, the methods further comprise the step of comparing the level of at least one metabolite from an individual to a periodontal disease metabolic profile.

In certain embodiments, the levels of oral cancer metabolite biomarkers are determined by using a technique selected from the group comprising HPLC, TLC, electrochemical analysis, capillary electrophoresis, mass spectrometry, refractive index spectroscopy (RI), Ultra-Violet spectroscopy (UV), fluorescent analysis, gas chromatography (GC), radiochemical analysis, Near-InfraRed spectroscopy (Near-IR), Nuclear Magnetic Resonance spectroscopy (NMR), and light scattering analysis (LS). In a specific embodiment, the salivary metabolites are detected by capillary electrophoresis (CE) coupled with time of flight mass spectrometry (TOF-MS). In other embodiments, the metabolites are detected by tandem mass spectrometry (MS/MS).

In certain embodiments, the methods of the present invention comprise the detection or determination of the level or concentration of at least one salivary metabolite biomarker found in Table 2. In other embodiments, the methods comprise the detection at least about 2, 3, 4, 5, 6, 7, 8, 9, 10, 15, 20, 25, 30, 35, 40, or all of the metabolites found in Table 2.

In some embodiments, the present invention provides methods of classifying or differentiating a disease state in an individual. In certain embodiments, the methods comprise the steps of: (a) determining the level of at least one metabolite in a saliva sample from said individual; (b) comparing the level of said at least one metabolite to a first salivary metabolic profile for a first disease state; (c) comparing the level of said at least one metabolite to a second salivary metabolic profile for a second disease state; and (d) determining which metabolic profile most closely correlates with the level of said at least one metabolite, thereby classifying the disease state in said individual.

In one embodiment, the present invention provides methods of classifying or differentiating a specific type of oral cancer from another type of oral cancer. In other embodiments, the methods allow for classification or differentiation of oral cancer from periodontal disease or oral cancer from a non-oral cancer. In yet other embodiments, the methods provided herein allow for the classification or differentiation of one stage of a specific disease from a second stage of the same disease. For example, the methods of the invention allow for the classification or differentiation of a specific type of oral cancer at a first stage from a second, more advanced stage, of the same type of oral cancer, or of an oral cancer type with a first survival or metastasis prognosis from an oral cancer of the same type with a second survival or metastasis prognosis.

Many correlation methodologies may be employed for the comparison of both individual metabolite levels and metabolome profiles in the present invention. Non-limiting examples of these correlation methods include parametric and non-parametric methods as well as methodologies based on mutual information and non-linear approaches. Examples of parametric approaches include without limitation, Pearson correlation (or Pearson r, also referred to as linear or product-moment correlation) and cosine correlation. Non-limiting examples of non-parametric methods include Spearman's R (or rank-order) correlation, Kendall's Tau correlation, and the Gamma statistic. Each correlation methodology can be used to determine the level of correlation between the levels of individual metabolites in the data set. The correlation of the level of all metabolites with all other metabolites is most readily considered as a matrix. Using Pearson's correlation as a non-limiting example, the correlation coefficient r in the method is used as the indicator of the level of correlation. When other correlation methods are used, the correlation coefficient analogous to r may be used, along with the recognition of equivalent levels of correlation corresponding to r being at or about 0.25 to being at or about 0.5. The correlation coefficient may be selected as desired to reduce the number of correlated gene sequences to various numbers. In particular embodiments of the invention using r, the selected coefficient value may be of about 0.25 or higher, about 0.3 or higher, about 0.35 or higher, about 0.4 or higher, about 0.45 or higher, or about 0.5 or higher. The selection of a coefficient value means that where levels between metabolites in the data set are correlated at that value or higher, they are possibly not included in a subset of the invention. Thus in some embodiments, the method comprises excluding or removing (not using for classification) one or more metabolites that are present at a level in correlation, above a desired correlation coefficient, with another metabolite in the salivary data set. It is pointed out, however, that there can be situations of metabolites that are not correlated with any other metabolites, in which case they are not necessarily removed from use in classification.

In yet other embodiments, the present invention provides kits of the detection of salivary metabolites. In certain embodiments, the kits are for detection of salivary oral cancer metabolie biomarkers. In particular embodiments, the kits comprise at least one reagent that binds to salivary metabolite found in Table 2. In other embodiments, the kits comprise reagents that bind to at least about 2, 3, 4, 5, 6, 7, 8, 9, 10, 15, 20, 25, 30, 35, 40, or all of the metabolites found in Table 2.

### DEFINITIONS

“Metabolites” or “small molecules” include organic and inorganic molecules which are present in a biological sample, such as saliva or a cell. These include both products and intermediates of metabolism as well as catabolism. The term does not include large macromolecules, such as large proteins (e.g., proteins with molecular weights over 5,000, 6,000, 7,000, 8,000, 9,000, or 10,000 Da), large nucleic acids (e.g., nucleic acids with molecular weights of over 5,000, 6,000, 7,000, 8,000, 9,000, or 10,000 Da), or large polysaccharides (e.g., polysaccharides with a molecular weights of over 5,000, 6,000, 7,000, 8,000, 9,000, or 10,000 Da). The small molecules are generally found free in solution, in the cytoplasm of a cell, or in various organelles, such as the mitochondria, where they form a pool of intermediates which can be metabolized further or used to generate large molecules, called macromolecules. The term “small molecules” includes signaling molecules and intermediates in the chemical reactions that transform energy derived from food into usable forms. Examples of small molecules include sugars, fatty acids, amino acids, small polypeptides, nucleotides, small polynucleotides, intermediates formed during cellular processes, and other small molecules found in biological samples. In one embodiment, the small molecules of the invention are isolated.

The term “metabolome” may include all of the small molecules present in a given organism, biological fluid, such as saliva, biological sample, tissue, organ, cell, or subsets thereof. The metabolome includes both metabolites as well as products of catabolism. Metabolomes may also refer to subsets of small molecules found in a biological sample from an organism suffering from a disease, such as cancer or periodontal disease. In some embodiments, a metabolome or a disease metabolome may refer to subsets of small molecules from a biological sample, such as saliva, from an individual suffering from a disease, which vary in concentration as compared to those found in a similar biological sample from an individual not suffering from the disease. General methods for identifying a metabolome may be found, for example, is U.S. Pat. No. 7,005,255.

“Disease metabolome” refers to a set of metabolites present at different concentrations or levels in a biological sample from an individual or group of individuals suffering from a given disease. Disease metabolomes may be derived from a particular biological sample, i.e. saliva, tissue, or tumor types. Metabolome profiles may be generated from a single sample from an individual or multiple samples from an individual, or alternatively from one or more samples from a group of individuals. For example, a salivary oral cancer metabolome profile may be generated from samples of saliva taken from an individual or group of individuals suffering from oral cancer. In one embodiment, a salivary oral cancer metabolome profile of the present invention comprises levels of one or more metabolites found in Table 2. In other embodiments, a salivary oral cancer metabolome profile of the present invention may comprise the level of at least about 2, 3, 4, 5, 6, 7, 8, 9, 10, 15, 20, 25, 30, or all of the metabolites found in Table 2.

“Metabolome profile”, “metabolic profile”, “disease metabolome profile”, or “disease metabolic profile” all refer to the quantitative or qualitative level of metabolites found in a metabolome, such as a control or salivary metabolome, or disease metabolome, such as a salivary oral cancer metabolome or periodontal disease metabolome.

In one embodiment, the invention pertains to a small molecule profile of the entire metabolome of a species. In another embodiment, the invention relates to a disease metabolome or a subset thereof. In a particular embodiment, the invention relates to a salivary disease metabolome, such as a cancer metabolome or a periodontal metabolome. In other embodiments, the invention pertains to a salivary metabolome from a systemic or genetically predisposed disease. In another embodiment, the invention pertains to a computer database (as described below) of a metabolome from a species, e.g., an animal, e.g., a mammal, e.g., a mouse, rat, rabbit, pig, cow, horse, dog, cat, bear, monkey, or human. In another embodiment, the invention pertains to a small molecule library comprising a metabolome or a subset thereof from an organism, e.g., a mammal, e.g., a mouse, rat, rabbit, pig, cow, horse, dog, cat, bear, monkey, and, preferably, or human.

A “small molecule profile,” “metabolite profile,” or “metabolome profile” refers to information regarding the concentration level of one or more small molecules or metabolites. In some embodiments, the profiles of the present invention pertain to a disease metabolome or subset thereof, i.e. oral cancer metabolome, periodontal disease metabolome, systemic disease metabolome, or genetically predisposed disease metabolome, from a biological sample, i.e. saliva, blood, urine, biopsy, or tissue. The profiles of the present invention may be derived from a sample taken from a single individual, or alternatively from a cohort of individuals suffering from a disease.

The term “cancer” refers to human cancers and carcinomas, sarcomas, adenocarcinomas, lymphomas, leukemias, solid and lymphoid cancers, etc. Examples of different types of cancer include, but are not limited to, oral cancers, oral squamous cell carcinoma (OSCC), breast cancer, gastric cancer, bladder cancer, ovarian cancer, thyroid cancer, lung cancer, prostate cancer, uterine cancer, testicular cancer, neuroblastoma, squamous cell carcinoma of the head, neck, cervix and vagina, multiple myeloma, soft tissue and osteogenic sarcoma, colorectal cancer, liver cancer (i.e., hepatocarcinoma), renal cancer (i.e., renal cell carcinoma), pleural cancer, pancreatic cancer, cervical cancer, anal cancer, bile duct cancer, gastrointestinal carcinoid tumors, esophageal cancer, gall bladder cancer, small intestine cancer, cancer of the central nervous system, skin cancer, choriocarcinoma; osteogenic sarcoma, fibrosarcoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, and acute myelocytic leukemia. Cancers embraced in the current application include both metastatic and non-metastatic cancers.

As used herein, “oral cancer” refers to a group of malignant or neoplastic cancers originating in the head or neck of an individual. Non-limiting examples of oral cancers include cancers of the lip, tongue, throat, tonsils, neck, buccal vestibule, hard or soft palate, gums (including gingival and alveolar carcinomas), nasopharyngeal cancer, esophageal cancer, lingual cancer, buccal mucosa carcinoma, head and neck squamous cell carcinoma, and the like.

“Head and neck squamous cell carcinoma” refers to group of cancers of epithelial cell origin originating in the head and neck, including the oral cavity and pharynx. These tumors arise from diverse anatomical locations, including the oral cavity, oropharynx, hypopharynx, larynx, and nasopharynx, but in some cases can have in common an etiological association with tobacco and/or alcohol exposure. The oral cavity is defined as the area extending from the vermilion border of the lips to a plane between the junction of the hard and soft palate superiorly and the circumvallate papillae of the tongue inferiorly. This region includes the buccal mucosa, upper and lower alveolar ridges, floor of the mouth, retromolar trigone, hard palate, and anterior two thirds of the tongue. The lips are the most common site of malignancy in the oral cavity and account for 12% of all head and neck cancers, excluding nonmelanoma skin cancers. Squamous cell carcinoma is the most common histologic type, with 98% involving the lower lip. Next most common sites in order of frequency are the tongue, floor of the mouth, mandibular gingiva, buccal mucosa, hard palate, and maxillary gingiva. The pharynx consists of the oropharynx, nasopharynx, and hypopharynx. The most common sites of cancer in the oropharynx are the tonsillar fossa, soft palate, and base of tongue, followed by the pharyngeal wall. The hypopharynx is divided into the pyriform sinus (most common site of tumor involvement), posterior pharyngeal wall, and postcricoid region.

“Periodontal disease” refers to a group of diseases affecting the gums of an individual, including gingivitis, periodontitis, and the like. Periodontal diseases may be further classified as aggressive, chronic, or necrotizing. Periodontitis is generally characterized by inflammation of the periodontium tissues, including the gingiva, the cementum, the alveolar bone, and the periodontal ligaments.

“Therapeutic treatment” and “cancer therapies” refers to chemotherapy, hormonal therapy, radiotherapy, and immunotherapy.

By “therapeutically effective amount or dose” or “sufficient amount or dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Metastasis” refers to spread of a cancer from the primary tumor or origin to other tissues and parts of the body, such as the lymph nodes.

“Saliva” refers to any watery discharge from the mouth, nose, or throat. For the purposes of this invention, saliva may include sputum and nasal or post nasal mucous.

“Providing a prognosis” refers to providing a prediction of the likelihood of metastasis, predictions of disease free and overall survival, the probable course and outcome of cancer therapy, or the likelihood of recovery from the cancer, in a subject.

“Diagnosis” refers to identification of a disease state, such as cancer or periodontal disease, in a subject. The methods of diagnosis provided by the present invention can be combined with other methods of diagnosis well known in the art. Non-limiting examples of other methods of diagnosis include, detection of known disease biomarkers in saliva samples, oral radiography, co-axial tomography (CAT) scans, positron emission tomography (PE T), radionuclide scanning, oral biopsy, and the like.

The terms “cancer-associated metabolite,” or “tumor-specific metabolite,” or “biomarker,” or “metabolite biomarker,” interchangeably refer to a small molecule that is present in a biological sample, e.g. saliva, from a subject with a disease, such as cancer, periodontal disease, a systemic disease, or a genetically predisposed disease, at a different level or concentration in comparison to a biological sample from a subject without the disease, and which is useful for the diagnosis of the disease, for providing a prognosis, or for preferential targeting of a pharmacological agent to an affected cell or tissue.

It will be understood by the skilled artisan that markers may be used singly or in combination with other markers for any of the uses, e.g., diagnosis or prognosis of a disease such as oral cancer or periodontal disease.

“Biological sample” includes sections of tissues such as biopsy and autopsy samples, and frozen sections taken for histologic purposes. Such samples include saliva, blood and blood fractions or products (e.g., serum, plasma, platelets, red blood cells, and the like), lymph and tongue tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methylphosphonates, 2-O-methyl ribonucleotides, peptide-nucleic acids (PNAs).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. Antibodies can be polyclonal or monoclonal, derived from serum, a hybridoma or recombinantly cloned, and can also be chimeric, primatized, or humanized.

### Diagnostic and Prognostic Methods

The present invention provides methods of diagnosing an oral cancer or periodontal disease by examining metabolites or small molecules that are present at differential levels or concentrations in saliva. Diagnosis involves determining the level of one or more metabolites of the invention in a patient and then comparing the level to a baseline or range. Typically, the baseline value is representative of a metabolite of the invention in a healthy person or a individual not suffering from the disease of interest, as measured using a biological sample such as saliva or a tissue sample (e.g., tongue or lymph tissue), serum, or blood. Variation of levels of a metabolite of the invention from the baseline range (either up or down) indicates that the patient has the disease or is at risk of developing the disease or a metastatic form thereof, or extracapsular spread.

A person of ordinarily skill in the art will be able to determine the appropriate metabolite profile for the methods of the present invention by comparing small molecule or metabolite profiles from diseased subjects with healthy or control individuals. These comparisons can be manual, e.g., visually, or can be made using software designed to make such comparisons, e.g., a software program may provide a secondary output which provides useful information to a user. For example, a software program can be used to confirm a profile or can be used to provide a readout when a manual comparison between profiles is not possible. The selection of an appropriate software program, e.g., a pattern recognition software program, is within the ordinary skill of the art. An example of such a program is Pirouette. It should be noted that the comparison of the profiles can be done both quantitatively and qualitatively.

Metabolite profiles can be determined by many methods well known in the art. In one embodiment, a small molecule profile may be determined by using capillary electrophoresis (CE) and mass spectrometry (MS) (Garcia et al., Curr Opin Microbial., 11(3):233-39 (2008)), HPLC (Kristal, et al., Anal. Biochem., 263:18-25 (1998)), thin layer chromatography (TLC), or electrochemical separation techniques (see, WO 99/27361, WO 92/13273, U.S. Pat. Nos. 5,290,420, 5,284,567, 5,104,639, 4,863,873, and U.S. Pat. No. RE32,920). Other techniques for determining the presence of small molecules or determining the identity of small molecules of the cell are also included, such as refractive index spectroscopy (RI), Ultra-Violet spectroscopy (UV), fluorescent analysis, radiochemical analysis, Near-InfraRed spectroscopy (Near-IR), Nuclear Magnetic Resonance spectroscopy (NMR), Light Scattering analysis (LS), time of flight mass spectrometry (TOF-MS) and other methods known in the art.

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used (see, e.g., Self et al., Curr. Opin. Biotechnol., 7:60-65 (1996)). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Biomed Sci., 699:463-80 (1997)). Liposome immunoassays, such as flow-injection liposome immunoassays and liposome immunosensors, are also suitable for use in the present invention (see, e.g., Rongen et al., J Immunol. Methods, 204:105-133 (1997)). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)).

Specific immunological binding of the antibody to an epitope can be detected directly or indirectly. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent antibody specific for the epitope marker is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as polystyrene beads, magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

Useful physical formats comprise surfaces having a plurality of discrete, addressable locations for the detection of a plurality of different biomarkers. Such formats include microarrays, or “metabolite chips” (see, e.g., Ng et al., J Cell Mol. Med., 6:329-340 (2002)) and certain capillary devices (see, e.g., U.S. Pat. No. 6,019,944). In these embodiments, each discrete surface location may comprise a binding reagent to immobilize one or more metabolite markers for detection at each location. Surfaces may alternatively comprise one or more discrete particles (e.g., microparticles or nanoparticles) immobilized at discrete locations of a surface, where the microparticles comprise binding reagents to immobilize one or more metabolite markers for detection.

The analysis can be carried out in a variety of physical formats. For example, the use of microtiter plates or automation could be used to facilitate the processing of large numbers of test samples. Alternatively, single sample formats could be developed to facilitate diagnosis or prognosis in a timely fashion.

### Compositions, Kits and Integrated Systems

The invention provides compositions, kits and integrated systems for practicing the assays described herein using metabolites of the invention, binding reagents specific for metabolite biomarkers of the invention, etc.

The invention provides assay compositions for use in solid phase assays; such compositions can include, for example, one or more metabolite or one or more binding reagents for the metabolites of the invention immobilized on a solid support, and a labeling reagent. In each case, the assay compositions can also include additional reagents that are desirable for binding. Modulators of expression or activity of metabolites of the invention can also be included in the assay compositions.

The invention also provides kits for carrying out the diagnostic assays of the invention. The kits typically include one or more probes that comprise binding reagents that specifically bind to metabolites of the invention, and a label for detecting the presence of the probe. The kits may include several binding reagents specific for the metabolites of the invention.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical images.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen are sampled to obtain light intensity readings for each position. Multiple pixels are processed in parallel to increase speed. The apparatus and methods of the invention are easily used for viewing any sample, e.g., by fluorescent or dark field microscopic techniques.

### EXAMPLES

### Example 1

This example describes a large-scale metabolome analysis of salivary samples from disease patients suffering from an oral cancers (n=69) or periodontal disease (n=11) and 87 healthy control individuals.

Salivary metabolites of oral cancer (n=69) and periodontal diseases (n=11), and control (n=87) were analyzed by coupling capillary electrophoresis with electrospray ionization time-of-flight mass spectrometry (CE-TOF-MS). The biomarkers identified in the present example can be used for diagnosing or providing a prognosis for oral cancer.

Patient Selection

Salivary fluid from oral cancer patients and healthy control individuals was obtained from subjects of Caucasian, Asian, African-American, and Hispanic origins. The ethnicity, age, and the period of time between the saliva collection and measurement for the study cohort is summarized in Table 1. To show that the metabolites of the present invention can discriminate for oral cancer, and are not just cancer-specific, the levels of metabolites that were significantly different between the saliva of oral cancer patients and control individuals were also compared in patients suffering from breast and pancreatic cancer.

TABLE 1
Clinical variables for individuals included in the control and
disease cohorts.

| | | | Oral | Breast | Pancreatic | Periodontal |
|---|---|---|---|---|---|---|
| Ethnicity | | Control | cancer | Cancer | Cancer | diseases |
| Caucasian | | 37 | 41 | | | |
| Asian | | 15 | 5 | | | |
| African-American | | 12 | 4 | | N/A | |
| Hispanic | | 5 | 5 | | | |
| Missing | | 18 | 14 | | | |
| (Total) | | 87 | 69 | | | |
| Age: | Min-Max | 20-75 | 34-87 | 29-77 | 11-87 | 23-76 |
| | (Ave.) | (41.4) | (60.7) | (55.1) | (64.9) | (57.4) |

| | | | | | |
|---|---|---|---|---|---|
| Missing | 2 | 5 | 10 | 2 | 2 |

Saliva Collection

Subjects were asked to refrain from eating, drinking, smoking or using oral hygiene products for at least 1 hour prior to collection. Subjects were asked to rinse mouth with water. Five minutes after oral rinse, subjects were asked to spit into a 50 cc Falcon tube kept on ice, for example in a styrofoam coffee cup filled with ice. Subjects were reminded not to cough up mucus. Typically, five ml of un-stimulated saliva can be collected in 5-10 minutes. Saliva samples were then centrifuged at 2,600(×)g for 15 min at 4° C., and were spun another 20 min with the occurrence of incomplete separation. The supernatant was divided and transferred equally into 2 new tubes. Sample in one tube was used for protein analysis and supernatant in the second tube for RNA analysis. The cell pellet remained in the original tube, label with the same sample ID. Samples were processed and frozen within 30 minutes from the time of collection and then sent to the laboratory stored on dry ice.

Sample Preparation

Frozen samples were thawed and 27 μl of each was removed for analysis. 3 p. 1 of water containing 2 mM methionine sulfone and 2 mM 3-Aminopyrrolidine was added to each of the control and oral cancer salivary samples. A second set of salivary samples (17 controls, breast cancers, pancreatic cancers, and periodontosis samples) were thawed and 24 μl of each was used for analysis. 6 μl of water containing 1 mM methionine sulfone and 1 mM 3-Aminopyrrolidine was added to each of these sample.

Metabolite Standards

All chemical standards obtained from commercial sources were analytical or reagent grade and were prepared in Milli-Q (Millipore, Bedford, Mass.) deionized water, 0.1 N HCl or 0.1 N NaOH. Typical stock solutions were prepared at 1 mM, 10 mM, or 100 mM depending on the reagent. Working solutions were prepared just prior to use by diluting into deionized water to obtain the appropriate concentration.

Instrumentation

All CE-MS experiments were performed using an Agilent CE capillary electrophoresis system (Agilent technologies, Waldbronn, Germany), an Agilent G3250AA LC/MSD TOF system (Agilent Technologies, Palo Alto, Calif.), an Agilent 1100 series binary HPLC pump, the G1603A Agilent CE-MS adapter, and G1607A Agilent CE-ESI-MS sprayer kit. Data acquisition was performed using the G2201 AA Agilent Chemstation software for CE and the Analyst QS software (v. 1.1) for TOF-MS.

CE-TOF-MS Analysis

Metabolite separations were performed in a fused-silica capillary (50 μm diameter, 100 cm total length) filled with 1M formic acid as the electrolyte. Sample solutions were injected at a pressure of 50 mbar for 3 sec, and 30 kV voltage was applied. The capillary temperature was maintained at 20° C. and the sample tray was maintained below 5° C. Sheath liquid, consisting of a solution of methanol and water (50% v/v) containing 0.5 μM reserpine, was delivered at 10 μl/min. ESI-TOF-MS was performed in the positive ion mode. The capillary voltage was set to 4,000 V, and nitrogen gas (heater temperature 300° C.) was set at a flow rate of 10 psig. For the TOF-MS, the fragmentor, skimmer, and OCT RFV voltages were set at 75 V, 50 V, 125 V respectively. An automatic recalibration function was performed using two reference masses of reference standards. The methanol dimmer adduct ion ([2MeOH+H]⁺, m/z 65.059706) and hexakis phosphazene ([M+H]⁺, m/z 622.028963) provided the lock mass for exact mass measurements. Exact mass data were acquired at the rate of 1.5 cycles/sec over a 50-1,000 m/z range.

Metabolite Identification

Standard errors in mass accuracy of the metabolite measurement technique based on the normalization of standard mass calibration by CE-TOF-MS used in this study were 10 ppm in the range from 200 to 400 m/z and 30 ppm outside of this range (Soga, T. et al., J Biol Chem, 281(24):16768-76 (2006)). Given that the MS data alone, at this level of precision, does not allow for the assignment of metabolites to unknown peaks (Kind, T. and O. Fiehn, BMC Bioinformatics, 7:234 (2006)), salivary samples were either spiked with candidate molecules prior to analysis or tandem mass spectrometry (MS/MS) fragment spectrums were compared, if candidate compounds were commercially available. The Alanine, beta-Alanine, Aspartic acid, Betanine, Cadaverine, Carnitine, Citrulline, D-alpha-aminobutyric acid, gamma-Aminobutyric acid, Glutamic acid, Glutamine, Glycine, Hypoxanthine, Isoleucine, Leucine, Lysine, Ornitine, Pipecolic acid, Piperidine, Phenylalanine, Proline, Putrescine, Serine, Threonine, Tyrosine, Tryptophan, and Valine, were identified by based on the matched m/z values and normalized migration times of corresponding standard compounds. The composition formulae are also confirmed by isotope distribution pattern.

The other peaks were identified based on the m/z value and the predicted migration time by Artificial Neural Networks (ANNs) (Sugimoto, M. et al. Large-scale prediction of cationic metabolite identity and migration time in capillary electrophoresis mass spectrometry using artificial neural networks. Anal Chem 77 (1), 78-84 (2005)). The candidates compounds were obtained from Kyoto Encyclopedia of Gene and Genomics (KEGG) database (Goto, S. et al. LIGAND: database of chemical compounds and reactions in biological pathways. Nucleic Acids Res 30 (1), 402-404 (2002)) and Human Metabolome Database (HMDB) (Wishart, D. S. et al. HMDB: the Human Metabolome Database. Nucleic Acids Res 35 (Database issue), D521-526 (2007)). The theoretical and measured m/z, and measured and predicted migration times for Choline were 104.1070 m/z and 104.1075 m/z (4.96 ppm) and 8.75 min (parenthetic values are errors). and 7.78 min. (0.97 min.), respectively. For Ethanolamine, those were 62.0600 m/z and 62.0601 m/z (1.21 ppm) and 8.16 min. and 7.78 min. (0.38 min.), respectively. For Piperideine, those were 84.0808 m/z and 84.0807 m/z (0.42 ppm) and 8.78 min. and 7.84 min. (0.94 min.), respectively. For Pyrroline hydroxycarboxylic acid, those were 130.0499 m/z and 130.0498 m/z (0.230 ppm) and 12.90 min. and 13.09 min. (0.19 min.), respectively.

Most of the conditions were identical to those used in the cationic metabolite analysis using CE-TOF-MS. Methanol-water (50% v/v) containing 1 μM reserpine was delivered as the sheath liquid at 5 μl/min. ESI-Q-TOF-MS was conducted in the positive product ion scan mode; the ion spray voltage was set at 5,500V. Dry air (GS1) was maintained at 10 psi. The declustering potential 1 and 2, and the collision energy voltage ware set at 60V, 15V, and 20V, respectively. Recalibration was manually performed with reserpine ([M+H]^{÷}, m/z 609.2906) and its fragment ion ([M+H]+, m/z 195.0652).

Data Analysis and Results

Raw data were analyzed with software performing noise-filtering, baseline correction, peak detection, and integration of peak area from sliced electropherogram. The width of each electropherogram was fixed at 0.02 m/z. Similar software is commercially available, such as Mass Hunter from Agilent Technologies, or XCMS for LC-MS data (Smith, C. A. et al., Anal Chem, 78(3):779-87 (2006)). Subsequently, accurate m/z values for each peak were detected in time and calculated with Gaussian curve fitting to the mass spectrum on the m/z domain peak. The alignment of peaks in multiple measurements were performed with dynamic programming techniques described in (Baran, R. et al., BMC Bioinformatics, 7:530 (2006)) and generated overlaid electropherograms of matched peaks were generated.

Statistically significant peaks (p<0.15) from the salivary control and the oral cancer samples were aligned. All peak areas were standardized to internal controls resulting in relative areas for the normalization of signal intensities in order to avoid injection volume bias and sensitivity variance of the detector between multiple measurements. Peaks undetected within a threshold S/N ratio of 2, were considered to have a peak area of 0. The relative areas of the pancreatic cancer, breast cancer, periodontal disease, and second control cohort salivary samples were multiplied by a factor of 1.25/1.1 to account for the standardization of sample concentration.

CE-TOF-MS identified an average of 3041 peaks for each sample (Min. 1585, Max 8400, S.D. 1137). Isotopic compounds, ringing, spikes, fragment ions, and adduct ions were then removed and the peak data sets were compared across the sample profiles (87 control samples and 69 oral cancer samples). Peaks were aligned according to m/z and migration time. 45 metabolites were identified as showing significant differences in comparison with healthy controls (p<0.05; Steel-dwass test). The obtained marker pool for discriminating oral cancer and controls includes 28 metabolites; Pyrroline hydroxycarboxylic acid, Leucine with Isoleucine, Alanine, Choline, Tryptophan, Valine, Threonine, Pipecolic acid, Glutamic acid, Histidine, Taurine, Piperideine, Carnitine and other 2 metabolites (p<0.001; Steel-dwass test), and Piperidine, alpha-Aminobutyric acid, Phenylalanine and a metabolite (p<0.01; Steel-dwass test), also Betaine, Serine, Tyrosine, Glutamine, beta-Alanine, Cadaverine and other two metabolites (p<0.05; Steel-dwass test). The detected markers for the all cohorts are listed in Table 2.

TABLE 2
Identified salivary metabolites that discriminate between salivary samples from
healthy individuals and patients with oral cancers

| | | | control | | oral cancer | oral cancer |
|---|---|---|---|---|---|---|
| | | control | vs | oral cancer | vs | vs |
| | | vs | periodontal | vs | pancreatic | periodontal |
| Metabolite marker candidates | HMDB† | oral cancer | desease | breast cancer | cancer | desease |
| C₂H₆N₂ | | 0.260 | 3.46 × 10⁻⁴*** | 1.51 × 10⁻⁸*** | 2.34 × 10⁻⁶*** | 6.26 × 10⁻⁶*** |
| (59.0616 m/z) | | | | | | |
| C₃₂H₄₈O₁₃ | | 0.834 | 0.907 | 4.75 × 10⁻⁴*** | 0.137 | 0.444 |
| (214.444 m/z) | | | | | | |
| C₃H₇NO₂ | | 0.955 | 0.0242* | 0.154 | 3.68 × 10⁻⁴*** | 0.0662 |
| (90.0552 m/z) | | | | | | |
| C₄H₁₂N₅ | | 0.686 | 0.0251* | 0.323 | 0.00636** | 0.469 |
| (131.1174 m/z) | | | | | | |
| C₄H₉NO₂ | | 0.620 | 0.779 | 1.00 | 0.00504** | 0.995 |
| (104.0705 m/z) | | | | | | |
| Cadaverine | HMDB02322 | 0.0422* | 0.00100** | 0.993 | 0.449 | 0.488 |
| C₃₀H₆₂N₁₉O₂S₃ | | 0.367 | 0.0141* | 0.0354* | 0.0812 | 0.420 |
| (409.2312 m/z) | | | | | | |
| C₁₈H₃₄N₆O₆ | | 0.891 | 0.997 | 0.143 | 0.0247* | 0.914 |
| (215.1269 m/z) | | | | | | |
| alpha-Aminobutyric acid | HMDB00650 | 0.00256** | 0.00796** | 1.00 | 0.0543 | 0.811 |
| Alanine | HMDB00161 | 2.45 × 10⁻⁴*** | 0.00647** | 0.956 | 0.0968 | 0.945 |
| Putrescine | HMDB01414 | 0.890 | 0.0227* | 0.138 | 0.0399* | 0.444 |
| C₅H₁₄N₅ | | 0.0212* | 0.0825 | 0.998 | 0.471 | 0.994 |
| (145.1331 m/z) | | | | | | |
| Piperidine | | 0.00119** | 0.137 | 0.165 | 1.00 | 0.972 |
| Taurine | HMDB00251 | 3.57 × 10⁻⁵*** | 6.70 × 10⁻⁵*** | 6.82 × 10⁻¹⁰*** | 5.82 × 10⁻⁷*** | 1.10 × 10⁻⁶*** |
| Piperideine | | 2.83 × 10⁻⁴*** | 3.19 × 10⁻⁴*** | 2.79 × 10⁻⁶*** | 0.00226** | 2.91 × 10⁻⁵*** |
| Pipecolic acid | HMDB00070 | 1.87 × 10⁻⁴*** | 0.00948** | 3.47 × 10⁻⁴*** | 0.175 | 1.97 × 10⁻⁴*** |
| C₄H₉N | | 2.02 × 10⁻⁷*** | 0.949 | 5.62 × 10⁻⁴*** | 0.996 | 0.00766** |
| (72.0813 m/z) | | | | | | |
| C₈H₉N | | 2.64 × 10⁻⁵*** | 0.0385* | 1.02 × 10⁻⁸*** | 0.0123* | 2.94 × 10⁻⁴*** |
| (120.0801 m/z) | | | | | | |
| Pyrroline hydroxycarboxylic acid | HMDB01369 | 1.28 × 10⁻⁵*** | 0.176 | 1.36 × 10⁻⁵*** | 0.867 | 0.00267** |
| Betaine | HMDB00043 | 0.0162* | 0.576 | 0.0183* | 0.133 | 0.0668 |
| C₄H₇N | | 0.101 | 0.502 | 0.0349* | 0.853 | 0.0858 |
| (70.0655 m/z) | | | | | | |
| C₆H₆N₂O₂ | | 0.00270** | 0.127 | 0.948 | 0.0715 | 0.974 |
| (139.05 m/z) | | | | | | |
| Leucine + Isoleucine | HMDB00687 + | 1.56 × 10⁻⁵*** | 0.00150** | 1.00 | 7.44 × 10⁻⁴*** | 0.868 |
| | HMDB00172 | | | | | |
| Phenylalanine | HMDB00159 | 0.00333** | 0.0198* | 1.00 | 0.00351** | 0.961 |
| Tyrosine | HMDB00158 | 0.0253* | 0.0279* | 0.926 | 0.0286* | 0.969 |
| Histidine | HMDB00177 | 6.87 × 10⁻⁴*** | 0.246 | 0.805 | 0.698 | 0.997 |
| Proline | HMDB00162 | 0.968 | 0.0598 | 0.0299* | 0.0171* | 0.291 |
| Lysine | HMDB00182 | 0.0779 | 0.459 | 0.426 | 0.00513** | 1.00 |
| Glycine | HMDB00123 | 1.00 | 0.193 | 0.0753 | 0.0122* | 0.352 |
| Ethanolamine | HMDB00149 | 0.684 | 0.00187** | 0.597 | 2.34 × 10⁻⁴*** | 0.132 |
| gamma-Aminobutyric acid | HMDB00112 | 0.833 | 0.0133* | 0.897 | 0.00108** | 0.367 |
| Aspartic acid | HMDB00191 | 0.287 | 0.403 | 0.416 | 4.37 × 10⁻⁵*** | 0.980 |
| Valine | HMDB00883 | 7.31 × 10⁻⁵*** | 0.0138* | 0.990 | 0.00325** | 0.999 |
| Tryptophan | HMDB00929 | 6.13 × 10⁻⁵*** | 0.0113* | 0.229 | 0.0461* | 1.00 |
| beta-Alanine | HMDB00056 | 0.0407* | 0.268 | 0.842 | 0.156 | 0.999 |
| Glutamic acid | HMDB00148 | 4.95 × 10⁻⁴*** | 0.0757 | 1.00 | 0.00312** | 1.00 |
| Threonine | HMDB00167 | 1.18 × 10⁻⁴*** | 1.80 × 10⁻⁴*** | 1.00 | 3.08 × 10⁻⁴*** | 0.0829 |
| Serine | HMDB00187 | 0.0197* | 0.00699** | 0.846 | 1.16 × 10⁻⁴*** | 0.435 |
| Glutamine | HMDB00641 | 0.0327* | 0.111 | 0.975 | 0.00228** | 0.998 |
| Choline | HMDB00097 | 2.30 × 10⁻⁵*** | 0.0580 | 0.0115* | 0.871 | 0.983 |
| Carnitine | HMDB00062 | 7.60 × 10⁻⁴*** | 0.996 | 0.247 | 0.652 | 0.670 |
| Glycerophosphocholine | HMDB00086 | 0.287 | 0.0322* | 7.05 × 10⁻⁶*** | 7.33 × 10⁻⁴*** | 0.0138* |
| C₇H₈O₃S | | 0.962 | 0.0154* | 2.71 × 10⁻⁴*** | 0.0176* | 0.0307* |
| (173.0285 m/z) | | | | | | |
| C₄H₅N₂O₁₁P | | 0.0421* | 0.256 | 5.17 × 10⁻⁶*** | 0.628 | 0.808 |
| (288.9691 m/z) | | | | | | |

*p < 0.05,
**p < 0.01,
***p < 0.001

The salivary metabolite profiles obtained by CE-TOF-MS are intricate products affected by multiple pathways. Therefore, direct relationships between our experimental profiles and specific biological pathways are difficult to elucidate. Nevertheless, connections can be made between several of the identified oral cancer metabolite biomarkers and general cancer progression principles.

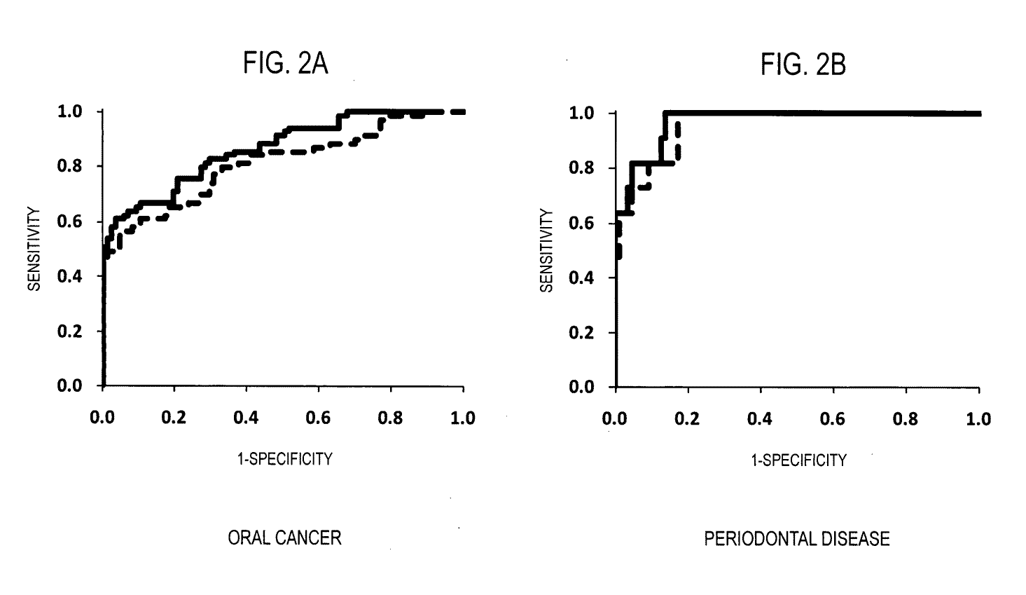

To evaluate the ability to discriminate oral cancer and periodontal disease from control samples with the identified biomarkers, multiple logistic regression (MLR) models were developed between the healthy cohort and each disease cohort independently. Step-wise variable selection method (backward procedure for eliminating the non-predictive peaks with threshold p>0.10) was used constructing the predictive model. The built models and the ones yielded by tenfold cross-validation procedure showed excellent separation abilities where all ROC values were more than 0.81 even though in cross validation result (FIG. 2 and Table 3).

TABLE 3
Logistic regression models of metabolome biomarkers for discriminating between
control and oral cancer or periodontal diseases.

| | | Coefficient | Standard | | Lower | Upper |
|---|---|---|---|---|---|---|
| Disease | Metabolite | value | error | P value | 95% CI | 95% CI |
| Oral | Intercept | 2.55 | 0.55 | <.0001 | 1.54 | 3.73 |
| cancer | Alanine | −26.43 | 13.31 | 0.047 | −53.84 | −1.95 |
| | Choline | −19.60 | 7.01 | 0.0052 | −34.20 | −6.49 |
| | Leucine + Isoleucine | −68.55 | 18.36 | 0.0002 | −108.66 | −36.72 |
| | Glutamic acid | −22.04 | 9.67 | 0.0226 | −42.63 | −4.73 |
| | 120.0801 m/z | −199.28 | 48.18 | <.0001 | −303.69 | −113.22 |
| | Phenylalanine | 63.86 | 16.17 | <.0001 | 35.11 | 98.99 |
| | alpha-Aminobutyric acid | 633.12 | 226.5 | 0.0052 | 212.09 | 1105.80 |
| | Serine | 67.50 | 28.92 | 0.0196 | 15.69 | 128.95 |
| Periodontal | Intercept | 2.87 | 0.93 | 0.0021 | 1.28 | 5.06 |
| diseases | Trimethylamine | −178.46 | 57.63 | 0.0020 | −325.59 | −87.53 |
| | Piperideine | 1276.4 | 713.65 | 0.074 | 311.48 | 3289.21 |

NOTE.
CI: confidence interval.

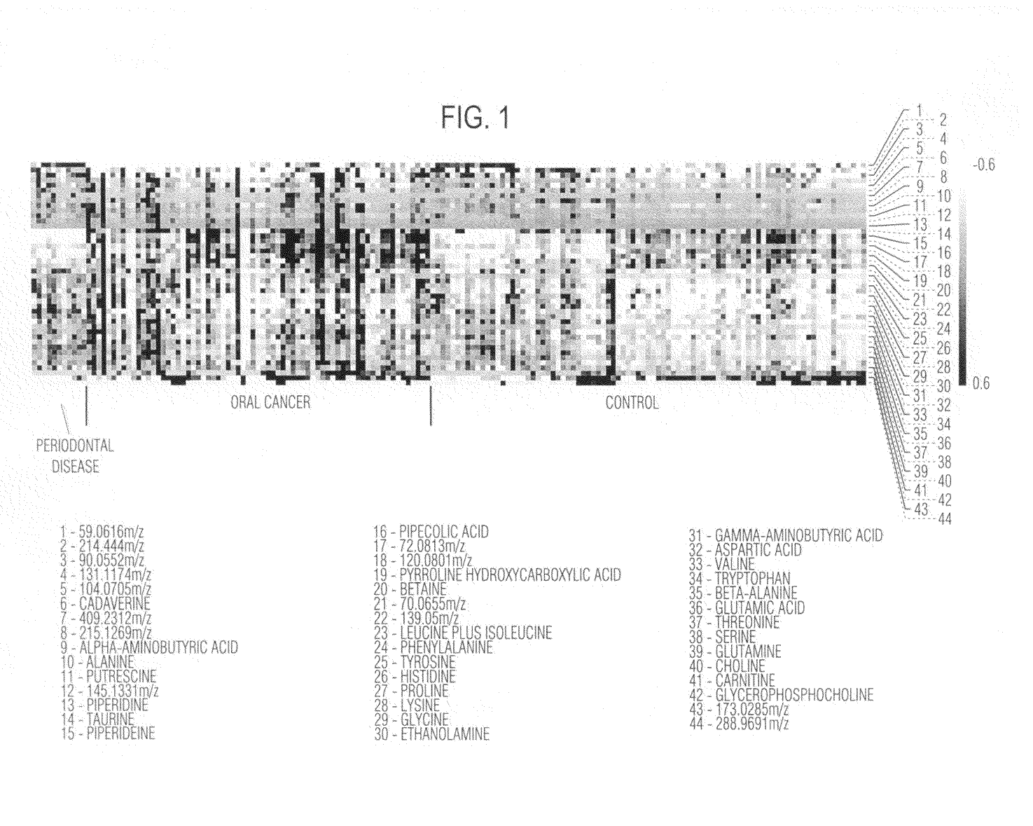

MLR models for periodontal disease yield high ROC values with only three metabolic markers. A metabolite heat map generated using the MeV TM4 software package (FIG. 1) revealed that levels of discriminatory metabolite biomarkers were constantly lower in the control and periodontal disease cohorts. Conversely oral cancers constituted widely diverse profile samples compared to the other groups, which help explain why the MLR models for oral cancer require more parameters for achieving accurate classification. The heterogeneous nature of oral cancer, including OSCC, oropharyngeal cancer, tongue cancer, or neck cancer, may cause such different profiles, which might deteriorate the separation capability of a single classification model.

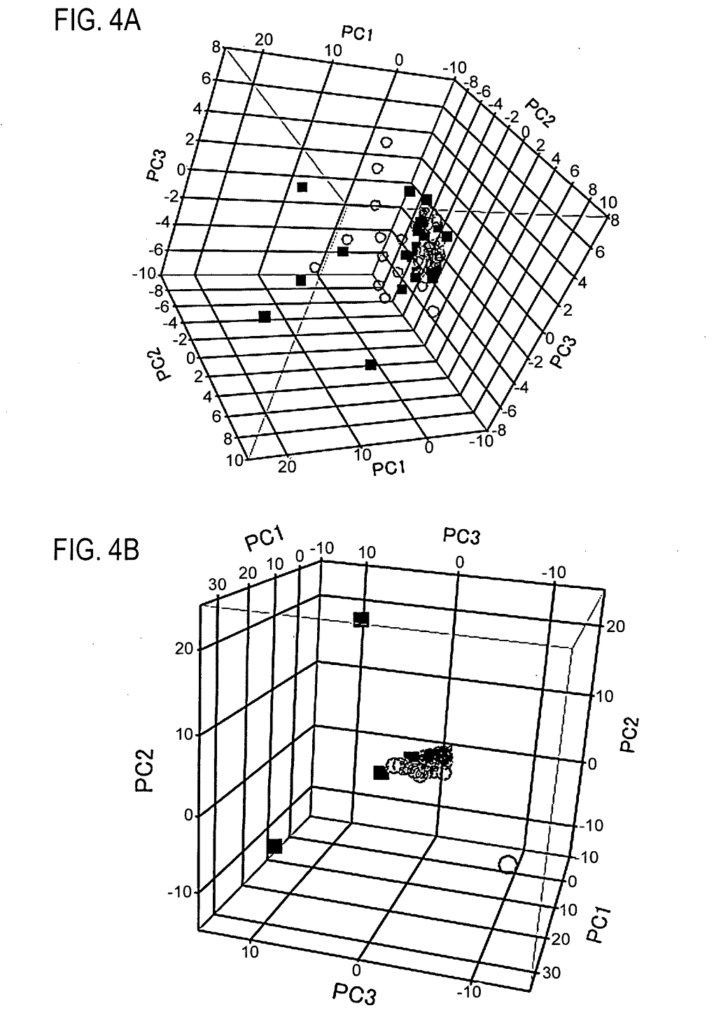

Principle component analysis based on each disease groups showed no clear prominent metabolite marker that has an enough ability to separate groups alone (FIG. 4). Although single metabolite is not enough to distinguish diseases, the changes of these marker candidates generally showed consistency with previous studies in the view of their changes. Polyamine is known generally correlated cell growth and proliferation (Casero, R. A., Jr. & Marton, L. J., Nat Rev Drug Discov 6 (5), 373-390 (2007); Gerner, E. W. & Meyskens, F. L., Jr. Nat Rev Cancer 4 (10), 781-792 (2004); Tabor, C. W. & Tabor, H. Annu Rev Biochem 53, 749-790 (1984)), also with tumour growth in oral cancer (Dimery, I. W. et al., Am J Surg 154 (4), 429-433 (1987)). Putrescine is used to monitor chemotherapy impact on oral cancer cells (Okamura, M. et al. Anticancer Res 27 (5A), 3331-3337 (2007)). The concentration of putrescine and cadaverine in serum decreased on radiotherapy in cancer patients, but were still higher than in healthy persons (Khuhawar, M. Y., et al., J Chromatogr B Biomed Sci Appl 723 (1-2), 17-24 (1999)). Oral polyamine levels are also affected by periodontitis and gum healing (Silwood, C. J., et al., J Dent Res 81 (6), 422-427 (2002)).

These results revealed that levels of ornithine and putrescine in the saliva of oral cancer patients were broadly higher than healthy controls. Although it was known that quantitative levels of polyamines were associated with regulating tumour growth and also periodontitis status, our results indicates that salivary polyamines are affected depending on cancer types and periodontitis, prominently higher levels in oral cancers than others.

In addition the polyamine, tryptophan (Carlin, J. M. et al. Experientia 45 (6), 535-541 (1989)), which was found higher in oral cancer, was previously identified as a marker for tumor development. As indirect correction among the detected peaks and human cancer, the repeat peptide of Pro-Pro-Gly, which showed higher levels in breast cancer, is known as an inhibitor of matrix metalloproteinase 2 (MMP-2, gelatinase A) which plays an important role in tumor invasion or metastatis (Jani, M. et al., Biochimie 87 (3-4), 385-392 (2005)). The expression levels of amino acid transporters ACST2 and LAT1 are elevated in primary human cancers, which cancer cells optimize their metabolic pathways by activating the exchange of amino acids between extra and intra cellular. Peptide and acid are derived from various sources, e.g. fragmented proteins, and the saliva metabolome profiles comprised of these compounds might have been the integrated results.

The identified polyamine containing metabolites ornithine, putrescine, and spermidine are all metabolites found in the urea cycle of hepatocyte cells. Putrescine is derived from ornithine by ornithine decarboxylase (ODC) and spermidine is derived from putrescine by spermidine synthase. Polyamines are known to play an important role in cell growth and proliferation (Gerner, E. W. and Meyskens, F. L., Jr., Nat Rev Cancer, 4(10):781-92 (2004); Takes, R. P., Oral Oncol, 40(7):656-67 (2004); Tabor, C. W. and Tabor, H., Annu Rev Biochem, 53:749-90 (1984); Seiler, N., Curr Drug Targets, 4(7):565-85 (2003); Casero, R. A., Jr. and Marton, L. J., Nat Rev Drug Discov, 6(5):373-90 (2007)). Inhibition of the polyamine synthesis pathway has been previously linked to tumor growth in oral cancers (Dimery, I. W. et al., Am J Surg, 154(4):429-33 (1987)). A number of anti-cancer polyamine complexes (including biogenic amines), with platinum(II) and palladium(II), have been widely used in chemotherapy (Lomozik, L. et al., Coordination Chemistry Reviews, 249(21-22):2335-2350 (2005)).

### Example 2

Several factors, including human papilloma virus (HPV), race, smoking, and alcohol dependence, were previously known as risk factors for the development of OSCC and oropharyngeal cancer (Kademani, D., Mayo Clin Proc, 82(7):878-87 (2007); Pintos, J. et al., Oral Oncol, 44(3): 242-50 (2008); D'Souza, G. et al., N Engl J Med, 356(19):1944-56 (2007)). Given the increased prevalence of oral cancers among older individuals, it has been speculated that age-related reductions in protective salivary antioxidant mechanisms and/or an age-related increases in exposure to oral carcinogens that cause DNA damage may play a role in the increased frequency of oral cancer (Hershkovich, O. et al., J Gerontol A Biol Sci Med Sci, 62(4):361-66 (2007)).

Age-related differences have been reported in a transcriptome study of the salivary gland (Srivastava et al. Archives of Oral Biology, 53 (11): 1058-1070 (2008)). It has been reported that other methods commonly used for standardization of metabolites in biofluid yield different statistical results (Schnackenberg et al. BMC Bioinformatics, 8: S3 (2007)); therefore, consistent decreases or increases in levels of metabolites among subjects with correlated clinical parameters should be accounted for. In the control subjects and patients with pancreatic cancer, there was a positive correlation between metabolites and age, whereas there was not in patients with oral or breast cancer or periodontal diseases. Accordingly, it is unlikely that age is correlated with the concentrations of salivary metabolites.

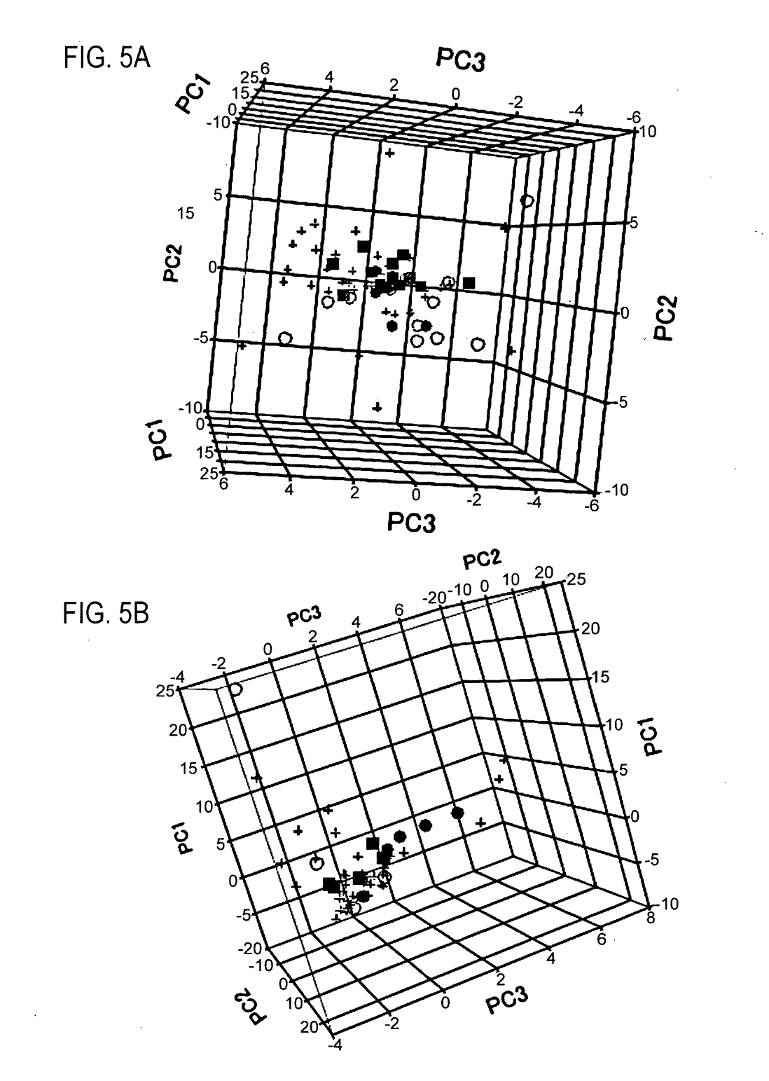

Relative areas of metabolite peaks were then compared between males and females in both the control cohort and the oral cancer cohort. In the oral cancer cohorts, piperidine, serine, and threonine levels were significantly different (p<0.05; Mann-Whitney) between males and females. In the healthy cohorts, tyrosine and the unidentified metabolite with a mass to charge ratio of 214.444 m/z was significantly different. The PCA analysis of 57 metabolite markers based on gender (FIG. 4) and race or ethnic groups (FIG. 5) also showed no clear separation, which might indicate the deviation of individual samples in available clinical parameters were also not significant.

Although, age had the greatest influence on metabolite concentrations in healthy individuals, metabolite concentrations in patients with oral cancers did not appear to be influenced by the individual's age. These data indicate that oral cancer status has a greater influence on metabolite concentration than does either age or sex.

### REFERENCES

1. Streckfus, C. F. & Bigler, L. R. Saliva as a diagnostic fluid. Oral Dis 8 (2), 69-76 (2002).
2. Soga, T. et al. Quantitative metabolome analysis using capillary electrophoresis mass spectrometry. J Proteome Res 2 (5), 488-494 (2003).
3. Soga, T. et al. Differential metabolics reveals ophthalmic acid as an oxidative stress biomarker indicating hepatic glutathione consumption. J Biol Chem 281 (24), 16768-16776 (2006).
4. Denny, P. et al. The Proteomes of Human Parotid and Submandibular/Sublingual Gland Salivas Collected as the Ductal Secretions. J Proteome Res 7 (5), 1994-2006 (2008).
5. Zimmermann, B. G. & Wong, D. T. Salivary mRNA targets for cancer diagnostics. Oral Oncol, doi:10.1016/j.oraloncology.2007.1009.1009 (2007).
6. Lawrence, H. P. Salivary markers of systemic disease: noninvasive diagnosis of disease and monitoring of general health. J Can Dent Assoc 68 (3), 170-174 (2002).
7. Kademani, D. Oral cancer. Mayo Clin Proc 82 (7), 878-887 (2007).
8. Epstein, J. B., Zhang, L., & Rosin, M. Advances in the diagnosis of oral premalignant and malignant lesions. J Can Dent Assoc 68 (10), 617-621 (2002).
9. Mao, L., Hong, W. K., & Papadimitrakopoulou, V. A. Focus on head and neck cancer. Cancer Cell 5 (4), 311-316 (2004).
10. Parkin, D. M., Bray, F., Ferlay, J., & Pisani, P. Global cancer statistics, 2002. CA Cancer J Clin 55 (2), 74-108 (2005).
11. Nieto, J., Grossbard, M. L., & Kozuch, P. Metastatic pancreatic cancer 2008: is the glass less empty? Oncologist 13 (5), 562-576 (2008).
12. Mignogna, M. D., Fedele, S., & Lo Russo, L. The World Cancer Report and the burden of oral cancer. Eur J Cancer Prev 13 (2), 139-142 (2004).
13. Sidransky, D. Emerging molecular markers of cancer. Nat Rev Cancer 2 (3), 210-219 (2002).
14. Nagler, R., Bahar, G., Shpitzer, T., & Feinmesser, R. Concomitant analysis of salivary tumor markers—a new diagnostic tool for oral cancer. Clin Cancer Res 12 (13), 3979-3984 (2006).
15. Shpitzer, T., Bahar, G., Feinmesser, R., & Nagler, R. M. A comprehensive salivary analysis for oral cancer diagnosis. J Cancer Res Clin Oncol 133 (9), 613-617 (2007).
16. Li, Y. et al. Salivary transcriptome diagnostics for oral cancer detection. Clin Cancer Res 10 (24), 8442-8450 (2004).
17. Hu, S. et al. Discovery of oral fluid biomarkers for human oral cancer by mass spectrometry. Cancer Genomics Proteomics 4 (2), 55-64 (2007).
18. Emekli-Alturfan, E. et al. Altered biochemical parameters in the saliva of patients with breast cancer. Tohoku J Exp Med 214 (2), 89-96 (2008).
19. Hu, S. et al. Salivary Proteomics for Oral Cancer Biomarker Discovery. Clin Cancer Res (2008).
20. Gerner, E. W. & Meyskens, F. L., Jr. Polyamines and cancer: old molecules, new understanding. Nat Rev Cancer 4 (10), 781-792 (2004).
21. Takes, R. P. Staging of the neck in patients with head and neck squamous cell cancer: imaging techniques and biomarkers. Oral Oncol 40 (7), 656-667 (2004).
22. Tabor, C. W. & Tabor, H. Polyamines. Annu Rev Biochem 53, 749-790 (1984).
23. Casero, R. A., Jr. & Marton, L. J. Targeting polyamine metabolism and function in cancer and other hyperproliferative diseases. Nat Rev Drug Discov 6 (5), 373-390 (2007).
24. Dimery, I. W. et al. Polyamine metabolism in carcinoma of the oral cavity compared with adjacent and normal oral mucosa. Am J Surg 154 (4), 429-433 (1987).
25. Okamura, M. et al. Induction of cell death by combination treatment with cisplatin and 5-fluorouracil in a human oral squamous cell carcinoma cell line. Anticancer Res 27 (5A), 3331-3337 (2007).
26. Khuhawar, M. Y., Memon, A. A., Jaipal, P. D., & Bhanger, M. I. Capillary gas chromatographic determination of putrescine and cadaverine in serum of cancer patients using trifluoroacetylacetone as derivatizing reagent. J Chromatogr B Biomed Sci Appl 723 (1-2), 17-24 (1999).
27. Byun, J. A. et al. Analysis of polyamines as carbamoyl derivatives in urine and serum by liquid chromatography-tandem mass spectrometry. Biomed Chromatogr 22 (1), 73-80 (2008).
28. Silwood, C. J., Lynch, E., Claxson, A. W., & Grootveld, M. C. 1H and (13)C NMR spectroscopic analysis of human saliva. J Dent Res 81 (6), 422-427 (2002).
29. Carlin, J. M. et al. Interferons and indoleamine 2,3-dioxygenase: role in antimicrobial and antitumor effects. Experientia 45 (6), 535-541 (1989).
30. Jani, M. et al. Hydroxamate-based peptide inhibitors of matrix metalloprotease 2. Biochimie 87 (3-4), 385-392 (2005).
31. Srivastava, A. et al. Age and gender related differences in human parotid gland gene expression. Arch Oral Biol. 53 (11), 1058-1070 (2008).
32. Schnackenberg, L. K. et al. Metabonomics evaluations of age-related changes in the urinary compositions of male Sprague Dawley rats and effects of data normalization methods on statistical and quantitative analysis. BMC Bioinformatics 8, S3 (2007).

It is understood that the examples and embodiments described herein are for illustrative purposes only and that various modifications or changes in light thereof will be suggested to persons skilled in the art and are to included within the spirit and purview of this application and scope of the appended claims. All publications, patents, and patent applications cited herein are hereby incorporated by reference in their entirety for all purposes.

What is claimed is:

1. A method of diagnosing or providing a prognosis for an oral disease in an individual, the method comprising the steps of:
(a) determining the level in a salivary sample from the individual of at least one salivary oral disease metabolite biomarker selected from those found in Table 2; and
(b) determining if the level of said at least one metabolite corresponds to a level found in an oral disease metabolome profile, thereby diagnosing or providing a prognosis for oral disease.

2. The method of claim 1, wherein said oral disease is oral cancer or periodontal disease.

3. The method of claim 1, wherein the level of said at least one salivary oral disease metabolite biomarker is determined by using a technique selected from the group comprising HPLC, TLC, electrochemical analysis, capillary electrophoresis, mass spectrometry, refractive index spectroscopy (RI), Ultra-Violet spectroscopy (UV), fluorescent analysis, gas chromatography (GC), radiochemical analysis, Near-InfraRed spectroscopy (Near-IR), Nuclear Magnetic Resonance spectroscopy (NMR), and light scattering analysis (LS).

4. The method of claim 1, wherein step (b) comprises the sub-steps of:
comparing the level of said at least one metabolite to a first profile comprising an oral disease metabolic profile;
(ii) comparing the level of said at least one metabolite to a second profile comprising a control metabolic profile; and
(iii) determining which metabolic profile is most similar to the level of said at least one metabolite from said individual, thereby diagnosing said individual as either having or not having an oral disease.

5. The method of claim 4, further comprising the step of comparing the level of said at least one metabolite to a third profile comprising a periodontal disease metabolic profile.

6. A method of identifying a salivary oral disease metabolite biomarker, the method comprising the steps of:
(a) determining the level of a metabolite in a first saliva sample from an individual with oral disease;
(b) comparing the level of said biomarker to the level of the same biomarker in a second saliva sample from an individual without oral disease; and
(c) determining if the level of the metabolite in said first sample is different from the level of the metabolite in said second sample, thereby identifying a salivary oral disease metabolite biomarker.

7. A kit for use in diagnosing or providing a prognosis for oral disease in an individual, the kit comprising at least one reagent for detecting a metabolite selected from those found in Table 2.
